(19) United States
(12) Patent Application Publication
(10) Pub. No.: US 2023/0051342 A1
(43) Pub. Date: Feb. 16, 2023

(54) PAIN MEDICATION MANAGEMENT SYSTEM

(71) Applicant: Fund for Medical Research Development of Infrastructue & Health Services Barzilai Medical Center (Re, Ashkelon (IL)

(72) Inventor: Omri Lubovsky, Mevaseret Zion (IL)

(21) Appl. No.: 17/783,656

(22) PCT Filed: Dec. 10, 2020
(86) PCT No.: PCT/IL2020/051280

§ 371 Date: Jun. 9, 2022

Related U.S. Application Data
(60) Provisional application No. 62/945,903, filed on Dec. 10, 2019.

(51) Int. Cl. G16H 20/10; G06F 3/01; G16H 50/20

(52) U.S. Cl. CPC G16H 20/10; G06F 3/013; G16H 50/20

(57) ABSTRACT

A pain management system for treating pain and/or detecting potential drug abuse in a patient suffering from pain, the system comprising at least one human machine interface (HMI), operable to acquire data generated by a patient responsive to pain that the patient experiences and data responsive to the patients intake of a drug for controlling the pain; at least one processor operable to process the pain and drug intake data to generate a pain control regimen; and at least one communication interface operable to support communications between an attending medical professional and the at least one HMI and/or the processor to enable the attending medical professional to access the pain and drug intake data and the pain control regimen

### RELATED APPLICATIONS

The present application claims the benefit under 35 U.S.C. 119(e) of U.S. Provisional Application 62/94,5903 filed on Dec. 10, 2019 the disclosure of which is incorporated herein by reference.

### TECHNICAL FIELD

Embodiments of the disclosure relate to monitoring and managing pain that a person experiences as a result of a medical condition.

### BACKGROUND

Managing and controlling pain is an overburdening economic and health crisis issue.

In the US alone over 100,000,000 people suffer from chronic pain, and cost to the US economy of pain management and direct and collateral pain damage to human activity is estimated to be between about 560 and 635 billion dollars per annum. Coordinating and administering the various pharmacological and non-pharmacological treatments available to deal with pain are complex, difficult to navigate tasks that are often inadequate to deal with an individual patient's pain and with the systemic pain management crisis of opioid addiction affecting modern, high population density societies. Opioid addiction affects public health as well as social and economic welfare, and the same problem is now being identified in other western countries as well. As of 2018, 130 people per day die in the US from overdose of opioids often prescribed for the treatment of acute pain.

### SUMMARY

An aspect of an embodiment of the disclosure relates to providing a personalized pain management system, also referred to as a “Pain-Watch system” or simply “Pain-Watch”, which comprises at least one human machine interface (HMI) configured to acquire for a same time period, real time samples of a person's experience of pain and of the person's interaction with a pain control regimen intended to alleviate the experienced pain. The samples include samples of explicit pain responses and samples of implicit pain responses that provide data, which may be referred to respectively as explicit and implicit pain data, relevant for determining quantitative measures of the person's experience of pain and the person's interaction with the pain control regimen. Explicit pain responses comprise volitional audio/visual behavior generated in response to explicit requests or challenges by the at least one HMI for information or performance of an activity relevant to pain and/or regimen interaction. Implicit pain responses comprise non-volitional behavior and behavior not solicited by the at least one HMI that is observed by the HMI and can be processed to provide real time measures relevant to pain experienced by the person and/or the person's interaction with a pain management regimen.

An optionally cloud based Pain-Watch hub processes the samples to provide at least one quantitative time resolved history, optionally referred to as a “pain history”, of the person's pain experience and at least one quantitative time resolved history, optionally referred to as a “regimen history”, of the person's interaction with the regimen. Optionally, the at least one pain history comprises an explicit pain history based on explicit pain data and/or at least one implicit pain history based on implicit pain data. Optionally, the Pain-Watch hub processes the pain histories to determine at least one correlation coefficient that indicates to what extent the explicit and implicit pain histories agree with each other and the explicit pain history reflects a reliable indication of the person's experience of pain. Optionally, the at least one pain history comprises a composite pain history comprising both explicit and implicit data, and optionally further comprising correlation between the data. A pain history comprising explicit and implicit data, and correlation between the data may also be referred to as “pain profile”. Similarly, the at least one regimen history may comprise an explicit, implicit, and/or composite regimen history. In case a correlation coefficient indicates disagreement between a pain history and a regimen history, this may be used, for example, as an indication of drug abuse. Optionally, the Pain-Watch hub processes the pain histories to determine at least one correlation coefficient that indicates to what extent the explicit and implicit pain histories agree with each other and the explicit pain history reflects a reliable indication of the person's experience of pain.

In an embodiment Pain-Watch hub comprises a database, which may be referred to as a Pain-Database of explicit and implicit data, which may be referred to respectively as explicit and implicit population data, optionally acquired for a population of people. Pain-Watch may process explicit and implicit data for a particular person responsive to the population data to provide the at least one quantitative time resolved pain and regimen histories for the particular person. In an embodiment the Pain-Watch hub employs an artificial intelligence (AI) to process explicit and implicit data acquired for a person to determine the at least one pain and at least one regimen histories for the person. Optionally, the hub uses population data in the database to provide training examples to train and/or update the AI.

In an embodiment Pain-Watch processes the at least one pain and regimen histories for a person to determine correlation between changes in the person's pain history and changes in the person's regimen history and efficacy of a given regimen associated with the regimen history in alleviating the person's pain. In an embodiment Pain-Watch comprises a dashboard with which a caretaker of the person may interact to view the at least one pain and at least one regimen histories.

This summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the disclosure are described below with reference to figures attached hereto that are listed following this paragraph. Identical features that appear in more than one figure are generally labeled with a same label in all the figures in which they appear. A label labeling an icon representing a given feature of an embodiment of the disclosure in a figure may be used to reference the given feature. Dimensions of features shown in the figures are chosen for convenience and clarity of presentation and are not necessarily shown to scale.

FIG. 1 schematically shows a Pain-Watch system operating to acquire samples of explicit and implicit data from a patient advantageous for managing the patient's pain, in accordance with an embodiment of the disclosure;

FIGS. 2A-2D schematically illustrate representative screenshots of HMIs in Pain-Watch Plan pages, in accordance with an embodiment of the disclosure;

FIG. 2E schematically illustrates a representative screenshot of a Pain-Watch Data page, in accordance with an embodiment of the disclosure; and

FIGS. 3A-3B show flow diagrams of Pain-Watch system modules for patient (FIG. 3A) and for attending medical professional (FIG. 3B), in accordance with an embodiment of the disclosure.

### DETAILED DESCRIPTION

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended. Wherever a general term in the disclosure is illustrated by reference to an example instance or a list of example instances, the instance or instances referred to, are by way of non-limiting example instances of the general term, and the general term is not intended to be limited to the specific example instance or instances referred to. The phrase “in an embodiment”, whether or not associated with a permissive, such as “may”, “optionally”, or “by way of example”, is used to introduce for consideration an example, but not necessarily a required configuration of possible embodiments of the disclosure. Unless otherwise indicated, the word “or” in the description and claims is considered to be the inclusive “or” rather than the exclusive or, and indicates at least one of, or any combination of more than one of items it conjoins

Described herein is a computer implemented system and method of treatment for personalized pain control regimen, which provides a patient with immediate attention for pain relief, while at the same time providing the attending medical practitioner with hands-on monitoring of patient's drug intake.

In an embodiment, a personalized pain control regimen is a regimen for pain management based on the patient's physical condition. In an embodiment, a patient's physical condition may be documented as information voluntarily provided by the patient, also referred to herein as explicit pain data, which is collected from explicit pain responses, or information acquired by the computer implemented system with regards to parameters identifiable and measurable by the system, for example as implicit pain responses. In an embodiment, the patient's physical condition may also be received in the system from information provided by the attending medical practitioner, for example medical data.

An aspect of an embodiment of the disclosure relates to providing a computer implemented pain management system for treating pain and/or detecting potential drug abuse in a patient suffering from pain, the system comprising: (i) at least one human machine interface (HMI), configured and operable to acquire and store raw log pain data of the patient's pain responses, to generate a pain history from the patient's pain data, and to provide the patient with a personalized pain control regimen in response to the pain data; (ii) at least one HMI configured and operable to acquire patient's report on drug intake; (iii) a processing unit configured and operable to process pain data obtained from patient pain responses; and optionally (iv) a computing device configured to receive data from the pain management system and provide an attending medical professional with accessibility to the patient's data.

In an embodiment, patient's pain responses may be explicit or implicit pain responses.

According to an embodiment, the computer implemented pain management system may be deployed in any computing system or device comprising a processing unit or processor and configured for implementing the pain management system described herein, including but not limited to a standalone computing device or system, a cloud computing-based system, or a computing distributed system.

In an embodiment, the HMIs described herein are configured in a personal computing device, such as a smartphone, cellular mobile device, a tablet, a personal computer, a desktop, a laptop, and any other personal computing device generally available to a user of the pain management system.

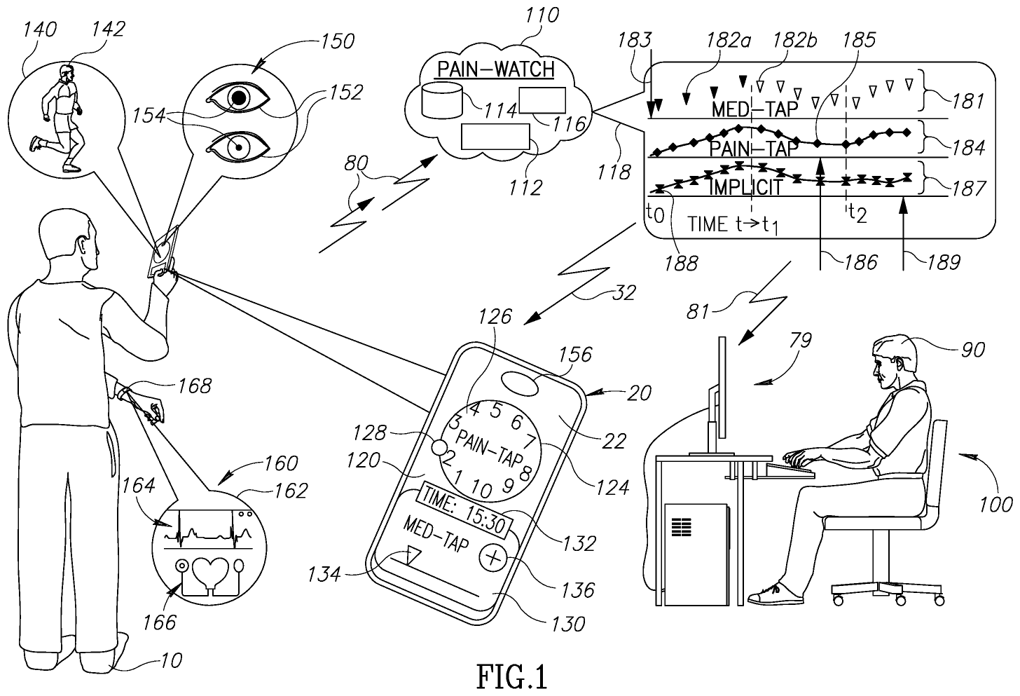

FIG. 1 schematically shows a Pain-Watch system 100 comprising, at least one HMI interacting with a patient 10 to acquire and process samples of explicit and/or implicit pain responses exhibited by the patient relevant for characterizing and/or managing pain that the patient experiences. Optionally, Pain-Watch system 100 comprises a cloud-based hub 110 to which the at least one HMI transmits data based on the samples for storage and processing to characterize and manage the pain. Hub 110 optionally comprises: (i) at least one communication interface 112 operable to communicate with patient 10, with the at least one HMI, and/or with an attending medical professional (AMP) 90 attending to the patient; (ii) a “Pain-database” 114 for storing data received from the at least one HMI; and (iii) a processor 116 for processing the data as discussed below.

As shown by way of example in FIG. 1, the at least one HMI comprises an explicit HMI 120 having a graphical user interface (GUI) displayed on a touch screen 22 of a mobile computing device such as a smartphone 20, and implicit HMIs for patient activity 140, pupil dilation HMI 150, cardiac function 160 and sweat (not shown). Patient activity HMI 140 is schematically represented by a running person icon 142, pupil HMI 150 is schematically represented by an image 152 of pupils 154 of a human eye, and cardiac HMI 160 is schematically represented by an image 162 comprising images of an electrocardiograph 164 and a blood pressure monitor 166.

In an embodiment explicit HMI 120 comprises any quantifiable physiological data, which may be acquired by the Pain-Watch system through access to a database of patient medical data, or it may be acquired by the Pain-Watch system through information provided by the patient.

In an embodiment patient medical data may be medical data entered in the system by the attending medical practitioner.

In an embodiment the GUI comprises a Pain-Tap icon 124, also referred to as Pain-Tap 124 and a Med-Tap icon 130, also referred to as Med-Tap 130.

Patient 10 may interact with Pain-Tap 124, by touch or visual gesture recognition, to indicate a level of pain severity that the person is experiencing, and/or to characterize the experienced pain.

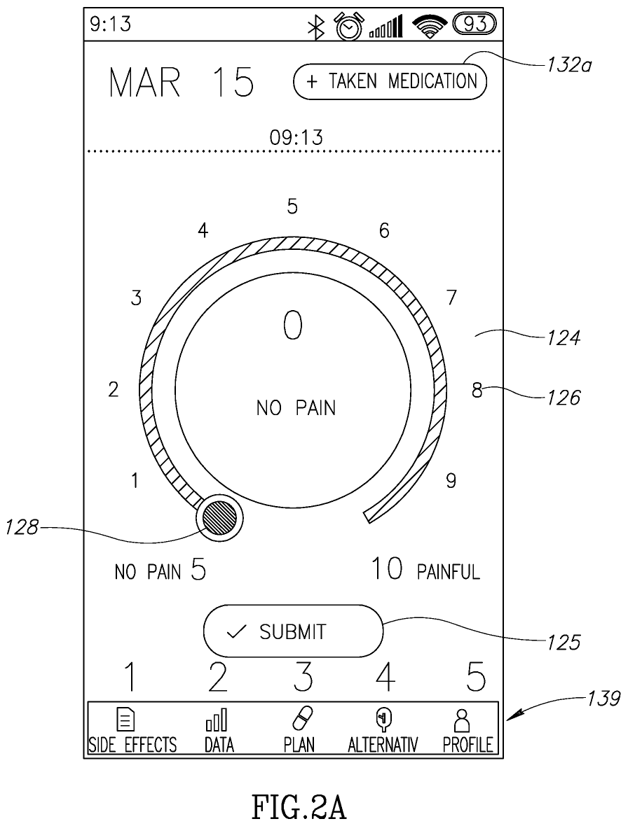

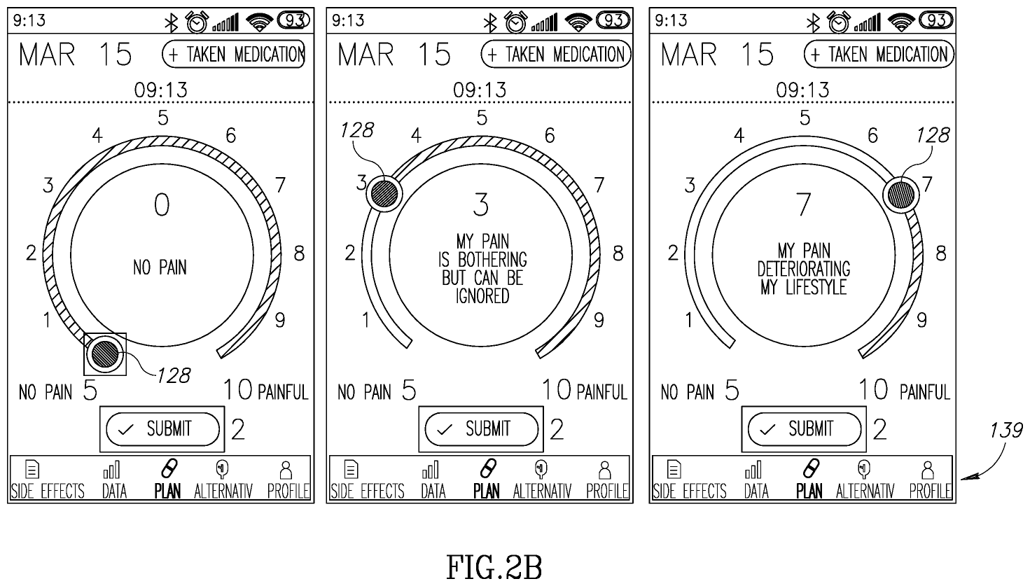

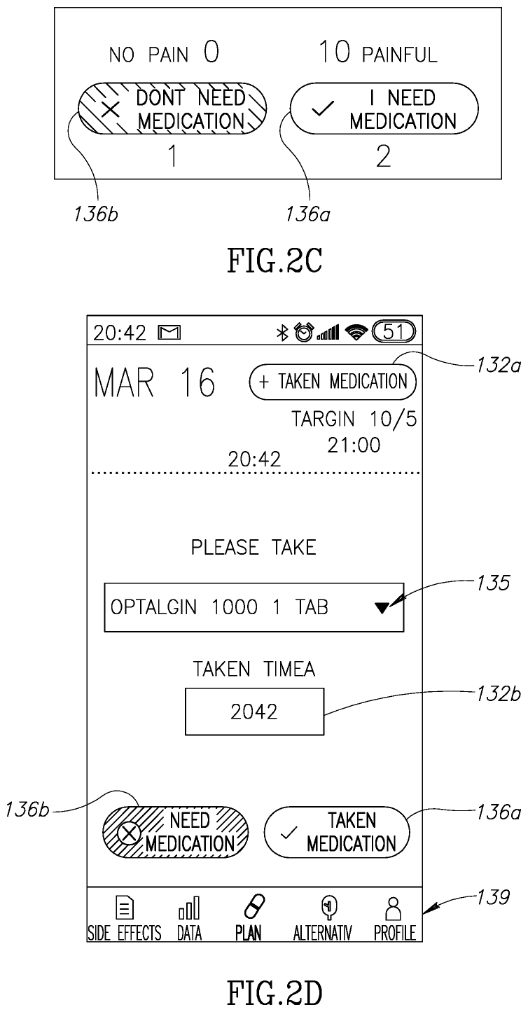

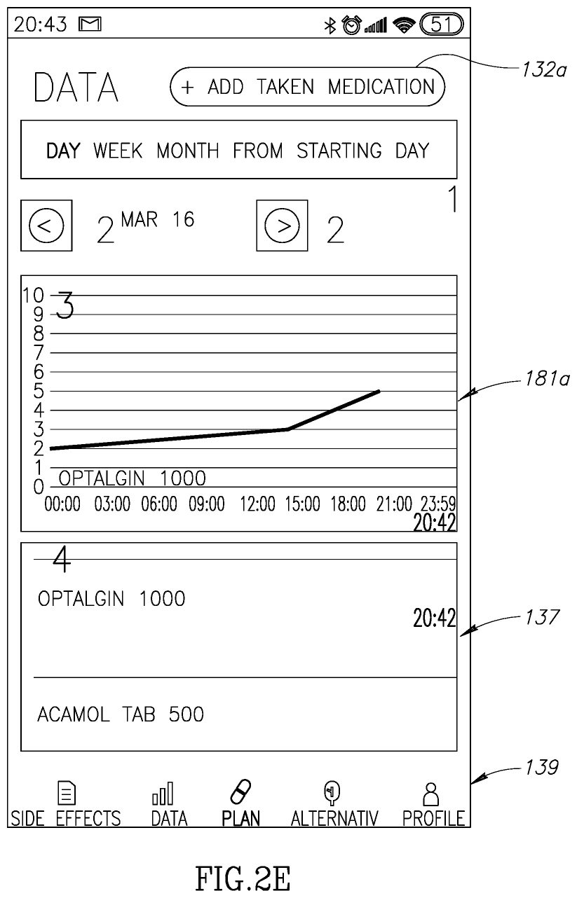

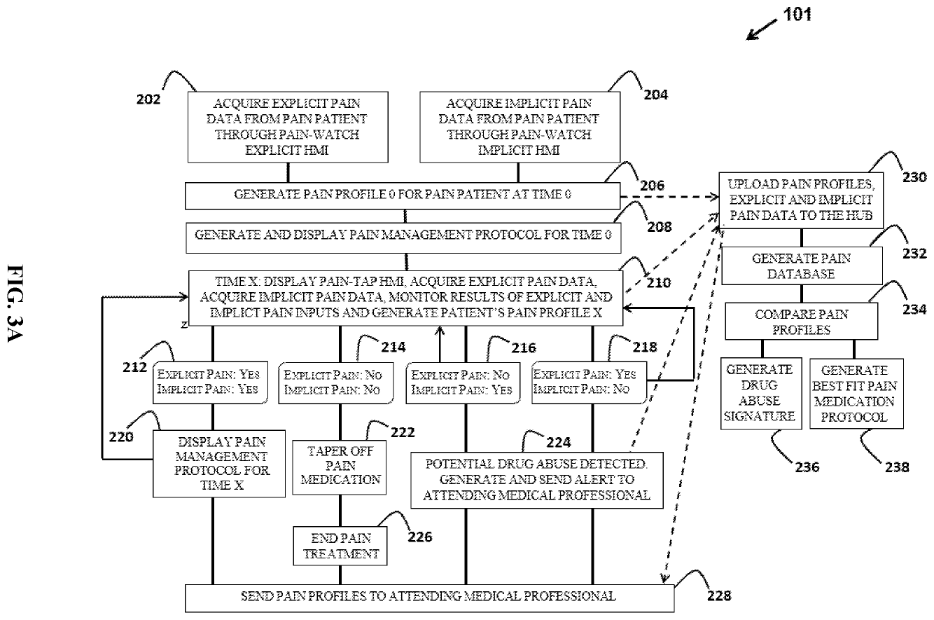

Pain-Tap 124 optionally displays a track 126 graduated with numbers 1-10 to indicate severity of pain, for which higher numbers indicate greater pain severity than lower numbers, and a slider 128 manually moveable along track 126. In an embodiment track 126 may be a circular track. Patient 10 may operate Pain-Tap 124 to indicate severity of pain by sliding slider 128 along track 126 to a numeral 0-10, relating to a numerical pain scale, indicative of the severity of pain the patient experiences. By way of example, a numerical pain scale along track 126 usable by explicit HMI 120 in accordance with an embodiment is defined in Table 1 presented below in Example 2. In an embodiment, Pain-Tap 124 displays a descriptive title of a level of pain severity corresponding to a pain number to which the slider is moved. FIGS. 2A-2B schematically show enlarged images of Pain-Tap 124 that exhibit details of Pain-Tap in accordance with an embodiment of the specification in which descriptive pain titles for different levels of pain are displayed (FIG. 2A, 2B) optionally inside track 126. Pain-Tap 124 may comprise a submit button 125, which pain patient 10 may push to enter a report on pain severity.

Patient 10 may interact with Med-Tap 130 to indicate times at which patient 10 takes a prescribed medication to control pain, dosage of the medication and requests for additional medication and/or change in medication dosage.

In an embodiment, Med-Tap 130 comprises reporting button 132 (FIG. 1) which patient 10 may touch to indicate times at which the patient takes a prescribed medication of a pain control regimen for controlling pain. Reporting button 132 optionally comprises a clock, for example a digital clock. Med-Tap optionally comprises a sliding scale 134 to indicate dosages of the medication respectively taken at indicated medication times. Optionally, Med-Tap 130 comprises a medication renewal button 136 that patient 10 may press to request renewal of a prescription for the pain medication that the patient is taking to manage pain.

FIG. 2C schematically shows an enlarged image of an embodiment of Med-Tap 130, comprising touch buttons 136a and 136b that patient 10 may touch for requesting medication or not, respectively, in accordance with an embodiment of the disclosure.

FIG. 2D shows an enlarged image of an embodiment of Med-Tap 130 that schematically exhibits details of Med-Tap, in accordance with an embodiment of the specification. Med-Tap 130 comprises reporting buttons 132a and 132b, for reporting drug intake and time, respectively; drug button 135 indicating drug regimen, comprising name of drug and dosage, and optionally a drop-down list of alternative or equivalent drugs; and touch buttons 136a and 136b described above.

Pupil HMI 150 is operable to image a pupil of patient 10 for use in tracking pupil sizes of patient 10 as implicit indications of severity of pain that patient 10 experiences. Pupil HMI 150 comprises an imaging system represented by a collecting lens 156 of smartphone 20 (FIG. 1) and a controller (not shown) that operates the imaging system to acquire and process images of a pupil of patient 10. Pupil HMI 150 may comprise face recognition software configured to determine when patient 10 is facing smartphone 20 in a direction that is advantageous for acquiring an image of a pupil of patient 10 for determining size of the pupil. The controller receives input from the face recognition software and when the input indicates that it is advantageous to image a pupil of patient 10, controls imaging system 156 to image the pupil. Pupil HMI 150 software may be configured to determine respective pupil sizes for the acquired pupil images and transmit pupil size data, optionally time stamped with acquisition times of the images, to hub 110 for processing by the hub. Pupil HMI 150 may further be configured to acquire other eye reactions related to pain, such as for example blinking rate.

Imaging system 156 may be configured to acquire additional pain-related facial reactions, such as image facial expressions/micro-expressions and/or facial coloration. Implicit data comprising such facial reactions may be acquired through pupil HMI 150, or through a designated facial HMI.

In an embodiment implicit activity HMI 140 acquires measurements of physical activity of patient 10. In an embodiment activity HMI 140 may determine measures of physical activity exhibited by patient 10 by determining, for example, how many steps patient 10 has taken, how many flights of stairs the patient climbed, and/or distance the patient traveled by walking and/or running, per unit time. Optionally, activity HMI 140 may use an accelerometer, inertial measurement unit (IMU), and/or GPS receiver comprised in smartphone 20 to determine measures of activity for patient 10.

Implicit Cardiac HMI 160 optionally comprises a sensor bracelet 168 which may for example be a component of a wristwatch comprising a sensor (not shown) that is responsive to heartbeats and a sensor (not shown) responsive to blood pressure. In an embodiment sensor bracelet 168 comprises a wireless communication interface, such as a WiFi or Bluetooth interface, that transmits implicit data based on heartbeat and blood pressure samples that the sensors in bracelet acquire to smartphone 20 for uploading, optionally to hub 110 for processing.

A sweat HMI for detecting and/or measuring sweat may comprise an electrodermal activity (EDA) sensor (not shown), which may for example be a ring comprising sensors to reliably monitor skin conductance (electrodermal activity and/or galvanic skin response).

As noted above, in an embodiment Pain-watch 100 comprises at least one explicit HMI and at least one implicit HMI.

Implicit pain data acquired by implicit HMIs may be optionally time stamped with acquisition times, similar to the time stamping by the pupil HMI mentioned above.

In an embodiment, implicit pain data may be acquired from other physical signs and symptoms correlated to pain that may be displayed by patient 10 and may be measured by designated HMIs, such as for example speech features such as speech rapidity or clarity of pronunciation, stuttering, piloerection (gooseflesh), hyperreflexia, vasoconstriction (cold, clammy hands), and diarrhea, amongst others.

In some embodiments, the acquired data may exhibit a positive correlation between implicit and explicit pain responses. A positive correlation between implicit and explicit pain responses may reflect the reported and detected presence of both implicit and explicit pain. In response to the presence of implicit and explicit pain, the system provides a pain management protocol, comprising details of type of drug to be taken, dosage and frequency of intake.

The attending medical practitioner may have access to the system at any given time, and thus monitor the patient's pain management protocol as well as the patient's drug intake.

In an embodiment, the initial medical data on a pain patient received by the system originates from information entered by the attending medical practitioner (AMP). Upon entering medical data on a pain patient, the AMP may also select a first protocol to be followed by the pain patient. The protocol for pain management provided by the AMP may be sent through the system and received by the patient's personal computing device.

In an embodiment, the system may receive the patient's medical data and provide a protocol for pain management.

In an embodiment, generation of a pain management protocol may be effected by an algorithm that combines pain data information, such as level of pain, with patient medical information, such as medical data and/or physiological parameters, with types of drugs and dosage, for example as provided in Table 4.

A positive correlation between implicit and explicit pain responses may reflect the reported and detected absence of both implicit and explicit pain. When these parameters identify the absence of pain, the system provides a protocol for tapering off pain medication in order to end the pain treatment.

The acquired data may exhibit an aberrant correlation between implicit and explicit pain responses. When an aberrant correlation is detected by the system, either from the presence of explicit pain and absence of implicit pain, or the contrary, from the absence of explicit pain and presence of implicit pain, the system generates a drug abuse alert signal, which reaches a computing device accessible to the attending medical practitioner.

In an embodiment, Pain-Watch menu 139 comprises icons which connect to Pain-Watch landing pages (also referred to herein as Pain-Watch pages) arbitrarily named herein as Side Effects, Data, Plan, Alternative and Profile, which are schematically represented in FIGS. 2A, 2B, 2D and 2E and described below in more detail. Generally, Pain-Watch pages are interactive, and one icon may lead to a plurality of pages that may follow each other as a result of patient 10 interactions with the pages, through GUIs and/or icons.

Examples of Pain-Watch Plan pages are shown herein in FIGS. 2A-2D, as well as in HMIs Pain-Tap 124 and Med-Tap 130 displayed in FIG. 1 and have been described above. In an embodiment, Pain-Watch Plan pages provide a platform for a patient to report pain, through the Pain-Tap GUI described above and represented in FIGS. 1, 2A and 2B, and request medication as represented for example in FIG. 2C. Plan pages provide a platform for reporting medication intake, for example through the Med-Tap GUI described in above and depicted in FIG. 1, or for example through push buttons 136a and 132a illustrated in FIGS. 2C and 2D. Plan pages also provide a means for the system to display a pain treatment plan, as illustrated in FIGS. 2A-2D. Pain-Watch page Plan may comprise a GUI requesting pain patient's input on whether the patient is in need of medication or not, as schematically represented in FIG. 2C by pushbuttons 136a and 136b. A positive response to need for medication may trigger a new Plan page, exemplified in FIG. 2D, indicating a drug and its dosage (135) and time to take (132b).

In an embodiment, a Plan page may also be displayed in the patient's smartphone as an alert, or as a reminder, which may also be referred to as a “push notification” of times to take a pain medication, optionally in combination with dosage details.

By way of example, Pain-Watch page Side Effects provides a platform for a patient to report side effects resulting from pain drug intake; while Pain-Watch page Data provides a platform for displaying data acquired, for example drug intake 181a per time (FIG. 1), and drug management protocol 137, as illustrated in FIG. 2E.

Pain-Watch page Alternative provides a platform for the system to display a pain treatment plan without drugs, which may be implemented as a complementary treatment. Pain-Watch page Profile provides a platform for the system to collect information on the patient, according to an embodiment of the disclosure.

A Pain-Watch HMI in accordance with an embodiment of the disclosure may comprise or have suitable access to, generically referred to as comprising, any software and/or hardware resources required to support functionalities of the HMI. For example, resources that an HMI in accordance with an embodiment of the disclosure uses to support HMI functionalities may comprise resources that are exclusive and dedicated to the HMI and/or resources made available to the HMI by a physical and/or a software entity that hosts and/or communicates with the HMI. The resources may comprise physical sensors that generate signals responsive to patient interaction with the HMI and software resources for processing the signals that the sensors provide. For example, software resources that an HMI comprises and/or has access to may be used by way of example to process samples acquired by the HMI to time-stamp the samples, to convert analog sample data to digital sample data, and to configure sample data for transmission to hub 110, and/or to transmit data based on the sample data to the hub.

By way of example, explicit HMI 120, may comprise software (not shown) for processing patient interaction with the GUI. Patient 10 is schematically shown in FIG. 1 interacting with the GUI in HMI 120. HMI 120 is shown uploading, schematically represented by lightning bolt arrows 80, data to hub 110 that HMI 120 generates based on acquired samples of the patient's interactions with the GUI. Software that HMI 120 uses to process interactions of patient 10 with the GUI to generate explicit data and transmit the explicit data to hub 110, may be dedicated Pain-Watch software downloaded to smartphone 20 and/or software that is endemic to a smartphone. HMI 120 may comprise its own memory and/or processor for using the HMI software and/or have access to any of the hardware and software comprised in smartphone 20 for using the software.

As in the case of explicit HMI 120, implicit HMIs, for example HMIs 140, 150 and 160, may have their own respective dedicated software and/or hardware, and may have access to software and/or hardware comprised in smartphone 20 to process and transmit implicit pain data based on interactions of patient 10 with the HMIs to hub 110. Lightning bolt arrows 80 schematically represent smartphone 20 transmitting the implicit pain data to Pain-Watch hub 110, as well as, as mentioned before, transmission of explicit pain data provided by HMI 120 to the hub.

In an embodiment Pain-Watch 100 comprises at least one processor for processing intrinsic and/or extrinsic data acquired by at least one or any combination of more than one Pain-Watch HMIs. For example, the at least one processor may process data acquired by any one or by any combination of more than one of pain patient's device 20, Pain-Watch hub 110, and/or an attending medical professional (AMP) 90, and/or computing device 79 (FIG. 1).

As referred to herein, the processor or processing unit may also be a processing hub, comprising any combination of hardware and software components. In an embodiment the processing unit may be cloud-based, or it may be a virtual entity hub. The processing hub may optionally have a memory and a processor configured to support functionalities of the hub. Alternatively processing of the system may occur in a personal computing device having access to a database for analysis of the results, and accessible from an attending medical practitioner's computing device.

In an embodiment, Pain-Watch hub 110 is configured and operable to receive and store data it receives from explicit and implicit HMIs, for example HMIs 120, 140, 150, and 160 for patient 10 in database 114. Optionally database 114 is a database that stores similar implicit and explicit pain data from a population of pain patients, who may be Pain-Watch users. Pain-Watch hub 110 may process and analyze data received from the HMIs to generate at least one time resolved pain history for patient 10. By way of example, pain histories generated by Pain-Watch hub 110 may comprise pain histories shown in an inset 118 (FIG. 1): Med-Tap and Pain-Tap pain histories 181 and 184 based on explicit data received from HMI 120 and an Implicit pain history 187 based on implicit data received from at least one or any combination of more than one of activity HMI 140, pupil HMI 150 and/or cardiac HMI 160. In an embodiment Pain-Watch hub 110 transmits, as schematically indicated by lightning bolt arrow 81, the pain histories to a computing device (not shown) accessible to AMP 90. FIG. 1 schematically indicates AMP 90 studying the transmitted pain histories which are displayed on the monitor of computing device 79.

Pain-Watch dashboard (not shown) is displayed on the computing device 79, and presents data acquired by Pain-Watch HMIs, processed and/or unprocessed. Data acquired by Pain-Watch may be transmitted to Pain-Watch dashboard directly from pain patient smartphone 20, or indirectly through Pain-Watch hub 110. By way of example Pain-watch dashboard may display pain histories, such as pain histories 181, 184 and 187 shown in inset 118.

Med-Tap, Pain-Tap and implicit pain histories 181, 184 and 187 are shown as functions of time from an initial time to along a same arbitrary time scale.

By way of example, variations of drug dosage (Y-axis) along time (X-axis 183) are shown in Med-Tap history 181. Filled triangles 182a and empty triangles 182b represent two prescription drugs taken and reported by patient 10, at time intervals t₀ to t₁ and from t₁ onwards, respectively. Height of a triangle symbol 182a or 182b above X-axis 183 indicates dosage of a prescribed medication taken by patient 10 at a time point on the X-axis to which the triangle points. Pain-Tap reported explicit pain intensity (Y-axis), represented by diamond symbol 185, along time (X-axis 186) is shown in Pain-Tap history 184. Height of diamond symbol 185 above X-axis 186 indicates pain that patient 10 registers by using Med-Tap 130 at a time on abscissa 186 above which the diamond symbol is located. Implicit pain intensity (Y-axis), acquired by at least one implicit HMI and represented by hourglass symbol 188, along time (X-axis 189) is shown in Implicit history 187. Height of an hourglass symbol 188 indicates a measure of pain experienced by patient 10 determined from implicit data.

In an embodiment, implicit and explicit pain responses acquired and quantified by Pain-Watch as pain data, may have or may be given different weights when used in generating a pain patient pain profile. As time elapses through the pain treatment, and if the treatment is effective, pain profile at time t₀, which indicates pain, and pain profile at time t₁, t₂, which theoretically demonstrate a trend of less pain through time, should distance themselves, until pain profile at time X is equivalent to no pain.

In an embodiment, analysis of explicit and implicit pain data before and after drug intake generates a personalized pain signature for a pain patient. At the initial time points of Pain-Watch, a pain signature may comprise pain features from implicit and explicit pain data in positive correlation, reflecting pain patient's experience of pain. For example, pain histories 181, 184 and 187 shown in inset 118 of FIG. 1 present a positive correlation at time interval t₀-t₁, where pain intensity increases according to both implicit and explicit parameters, as well as dosage of drug intake. A positive correlation is still observed at time interval t₁-t₂, where pain intensity decreases according to both implicit and explicit parameters, as well as dosage of drug intake. From time point t₂ and onwards, a positive correlation is still observed between drug intake (181) and extrinsic pain data (184), whereas implicit pain history data points substantially are stagnate and are not more affected by drug intake (187).

In an embodiment, an AMP module of the Pain-Watch system menu is available in an

AMP computing device 79. Pain-Watch AMP menu comprises icons which connect to Pain-Watch AMP landing pages arbitrarily named herein as Patients and Protocols. At the Patients page the AMP may introduce patients and patients' data. At the Protocols page, AMP Pain-Watch system may generate a protocol or a set of protocols compatible with each patient. In an embodiment, the AMP system may provide the AMP with access to the patient's data generated in the patient's Pain-Watch module.

In an embodiment, the pain management system may calculate the amount of drug a patient has taken in a day. The system may further limit the amount of drug a patient takes per day, for example by limiting the prescriptions made available to the patient. The amount of drug calculated may be the overall amount of all drugs taken or it may be the amount of each category of drug, for example narcotics, non-opioid analgesics, nonsteroidal anti-inflammatory drugs, proton pump inhibitors, etc.

In an embodiment, the pain signature for a pain patient after some time following a pain control regimen is expected to trend towards lack of pain, with intrinsic and explicit pain data substantially maintaining a positive correlation.

An attending medical professional may monitor pain patient's explicit and implicit data, and/or pain patient's pain signature, at any time during patient's use of the Pain-watch system.

Aberrant correlations between implicit and explicit pain data may indicate drug abuse.

Similarly, an aberrant pain signature may indicate drug abuse.

The computer implemented pain management system may generate a drug abuse alert when aberrant implicit and explicit pain data correlations occur. Alternatively, or additionally, Pain-Watch system may generate a drug abuse alert when an aberrant pain signature is detected. A drug abuse alert is transmitted to the attending medical professional and indicates the potential of medication misuse.

In an embodiment, when a drug abuse alert is generated the Pain-Watch system halts its functions, or at least it halts the presentation of treatment protocols until an attending medical professional intervenes.

Artificial Intelligence (AI) processing of pain signature data may detect aberrant pain signatures and predict potential drug abusers in a population of pain patients. Alternatively, AI processing of pain signature data may generate drug treatment regimens to patients suitable to each patient upon comparing each patient data with the pain database.

AI processing of pain signature data may employ an algorithm, for example a classifier, in order to detect a pain signature and classify it as a signature of a drug abuser, or of a potential drug abuser. Any of various classifiers, may be used for identifying drug abuser or potential drug abusers, such as, but not limited to, linear classifiers, like logistic regression and Naive Bayes Classifier, Nearest Neighbor classifier, Support Vector Machines classifiers, Decision Trees classifiers, Boosted Trees classifiers, Random Forest classifier, Neural Networks classifiers, or any other machine learning classifier.

The use of the computer implemented pain management system may be adopted by the medical community, medical associations and the like, as well as by the authorities, as conditional for the prescription of narcotic drugs. Thus, in an embodiment the present disclosure provides a method of performing drug abuse control in a population of patients where treatment with substances that potentially trigger dependency is prescribed, for example treatment with opioids, cannabis, or any other drug, the method comprising implementing a pain management system as described herein to said population of patients. A population of patients where treatment with substances that potentially trigger dependency is prescribed is a population of patients where opioid, cannabis, or any other drug, is indicated, and it may include post-surgery patients, cancer patients, patients suffering from chronic pain, and patients to whom opioids or cannabis are the best recommended drugs as known to person skilled in the art.

An aspect of an embodiment of the disclosure provides a method for treatment of pain, the method comprising providing a Pain-Watch system to a patient suffering from pain, wherein upon following the steps of the Pain-Watch system as described in the disclosure, pain is decreased and patient ceases to suffer from pain.

An aspect of an embodiment of the disclosure provides a method for detecting potential drug abuse, the method comprising providing a Pain-Watch system to a patient suffering from pain, and monitoring the system for drug abuse alert, whereby if a drug abuse alert is triggered by a pain profile generated by the system for the patient, a potential drug abuse is detected in the patient and transmitted to the attending medical practitioner.

Throughout the disclosure, the terms “drug” and “medication” are used interchangeably.

### EXAMPLES

### Example 1: Pain-Watch System

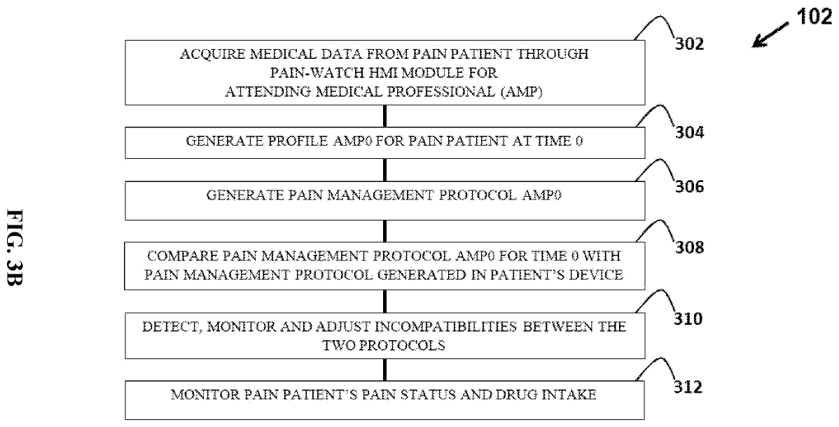

By way of example, operation of a Pain-Watch system in accordance with an embodiment is described with reference to FIGS. 3A-3B. FIG. 3A is a flow diagram 101 schematically illustrating steps of the computer implemented Pain-Watch 100 for the patient, with reference to elements depicted in FIGS. 1 and 2A-2E. FIG. 3B is a flow diagram 102 schematically illustrating steps of the computer implemented Pain-Watch 100 for the attending medical professional (AMP), in accordance with an embodiment of the disclosure.

Having a Pain-watch system 100 installed in a pain patient's mobile computing device, for example mobile phone 20, Pain-watch acquires through HMIs 120, and at least one of 140, 150 and 160, pain patient's explicit and implicit data, as described in blocks 202 and 204, respectively. Block 202 refers to acquisition of explicit pain data, through receiving input information that pain patient voluntarily provides to the system, through Pain-watch explicit HMIs pain tap 124 and med-tap 130. Additionally, or alternatively, pain patient may also input personal information through Pain-watch profile page. Additionally or alternatively, medical data on the patient may also be provided by an attending medical practitioner. Block 204 refers to acquisition of implicit pain data collected by implicit HMIs, for example pupil HMI 150 in pain patient's smartphone 20, or for example by a cardiac HMI in a wrist band, for example sensor bracelet 168, comprising detectors for pulse and heart rate. Following the initial data acquisition, Pain-Watch generates an initial pain profile, referred to as pain profile 0 in block 206, for pain patient. Pain profile 0 may be uploaded to Pain-Watch Hub 110 or directly to an AMP as indicated in block 228. Pain-Watch generated data moving from personal devices, such as smartphone 20 or an attending medical professional computer, to and from Pain-Watch hub are represented by dashed lines. Additionally or alternatively, pain profile 0 may be sent directly to a computing device available to an attending medical practitioner. Upon generating a pain profile 0, Pain-watch generates a pain management protocol or regimen 135 (FIG. 2D), and displays it in pain patient's computing device, in block 208, in order for it to be followed by pain patient. Time X, referred to in block 210, represents a time wherein a new entry of explicit and implicit pain data occurs. Time X is a time-point following a time interval X, when Pain-Tap 124 (FIG. 1-2C) is presented to pain patient 10 and acquires explicit data. Time interval X may be a time interval pre-programmed in the system or triggered by pain patient's interaction with any one of Pain-Tap 124, any Pain-Watch GUI or Pain-Watch page. Pain-Watch acquires explicit pain data provided by pain patient at said time X, for example by indicating pain level 0, 3 or 7, as schematically shown by slider 128 in FIG. 2B. Block 210 also refers to implicit pain data being acquired at time X through at least one implicit HMI. In block 210 Pain-Watch further monitors the results of explicit and implicit pain inputs acquired at time X, and generates a pain profile, pain profile X. Pain profile X may be uploaded to Pain-Watch Hub as indicated in block 228. Pain profile X provides information to Pain-Watch system which may reflect four possible outcomes, represented by outcome blocks 212, 214, 216 and 218. Additionally or alternatively, pain profile X may be sent directly to a computing device available to an attending medical practitioner

In block 212, the outcome of pain profile X is both explicit and implicit data indicating that the patient is experiencing pain. Pain-Watch displays a pain management protocol to be followed by pain patient, as indicated in block 220, such as for example a pain regimen shown in FIG. 2D, and the system returns to block 210, for acquiring explicit and implicit pain data and monitoring the results.

In block 214, the outcome of pain profile X is absence of pain by both explicit and implicit data. Pain protocol may detect the absence of pain and taper off pain medication, as described in block 222. As a result, pain treatment may be terminated, as indicated in block 226.

In block 216, the outcome of pain profile X is absence of pain by explicit data and presence of pain by implicit data, indicating that the patient may still be experiencing some level pain. Pain-Watch system returns to block 210, for acquiring explicit and implicit pain data and monitoring the results. This may indicate misuse of pain medication and send alert to attending medical professional, as indicated in block 224.

In block 218, the outcome of pain profile X is presence of pain by explicit data and absence of pain by implicit data. The system identifies the outcome as aberrant, which triggers the generation of an alert, described in block 224. This outcome is interpreted as potential drug abuse. Further, the alert triggered by the aberrant outcome is transmitted to the attending medical professional.

Outcomes are followed by sending pain profiles and data to the attending medical professional, as in block 228.

Data and profiles uploaded to the hub, in block 230, may be downloaded to a computing device used by, or made available to the use of, the attending medical professional, as shown by the dashed line from block 230 to block 228.

Data and profiles uploaded to the hub, in block 230, may generate a pain database, indicated in block 232. Pain database may be the source of pain data for sorting and comparing pain profiles, in block 234, and applying AI, for the generation of a drug abuse signature, in block 236. Furthermore, the pain database may apply AI on the pain data for the generation of optimized pain protocols for pain patients, as indicated in block 238.

Aberrant events in profile comparison, for example between intrinsic and extrinsic data, or between pain profile at time 0 and pain profile at time X may be indicative of drug abuse. An aberrant event is detected when there is lack of or negative correlation between explicit and implicit data. Generally, explicit and implicit data display a positive correlation, and are both indicative of the presence, or absence, of pain. If implicit data does not indicate pain, but explicit data does, it may generate a negative correlation between extrinsic and intrinsic data, if plotted for example for pain intensity. As shown in block 218, aberrant events will generate an alert, and the alert will be sent to the attending medical professional AMP in block 224.

By way of example, operation of Pain-Watch to support attending medical professional

(AMP) is schematically presented in a flow diagram 102 in FIG. 3B. In block 302, medical data from a pain patient is acquired by the system either by direct input from the AMP, or through communication with a central database of medical data, for example from a medical institution, or by a combination of the two input sources. In block 304, the AMP Pain-Watch module generates a profile AMPO for the pain patient, followed by the generation of an AMP pain management protocol, in block 306. The AMP pain management protocol generated at the AMP module is then compared, in block 308, with the protocol generated in the patient's Pain-Watch module, generated in block 208 of FIG. 3A. In block 310, incompatibilities between the two protocols are detected, monitored and adjusted, if there is a need. The AMP Pain-Watch system monitors pain patient's status and drug intake, by receiving the explicit and implicit pain inputs collected through the pain patient's Pain-Watch system.

The Pain-Watch system is configured to receive input from the patient in pain (pain patient), compute the information received and provide an output to the patient and/or to the AMP. At any timepoint, after starting a pain control regimen, it may be expected that a pain patient will present a lower reported pain on a pain scale (according to the medical condition of the patient), comparing to the initial pain reported. For example, on a numerical pain scale between 1-10, the pain should be closer to 0 than when the regimen started. The standard deviation of reported pain is expected to be as low as possible, within the timeline of the regimen, and a pain patient is expected to experience the minimum number of peaks of severe pain. The pain control regimens offered through Pain-Watch system are designed to provide a minimal amount of narcotics necessary for treating pain (regarding dosage and frequency) as possible, as well as to provide guidance to a pain patient on gradual reduction of medication intake, especially narcotics. It is expected that as the time passes after the patient starts feeling the pain, irrespective of the cause of pain, for example injury, surgery, or other acute pain, the intervals in between medications may increase and the use of narcotics may decrease. If this expected pattern is not observed, an alert of potential drug abuse or other medical problem may be generated by the Pain-Watch system. The pain control algorithm comprised in the Pain-Watch system is designed to recommend the minimal possible drug dosage for pain control to each patient. As the patient level of comfort improves, or as the pain level decreases, the pain control algorithm is designed to recommend lower amounts of pain killers.

### Example 2: Pain Scales

Pain description for the numerical pain scale exemplified in FIGS. 2A-2B is presented in Table 1.

TABLE 1
Pain-watch Numerical Pain Scale

| Number Scale | Pain Description |
|---|---|
| 0 | No Pain |
| 1 | Faint = Pain Barely Noticeable |
| 2 | Mild = Pain Light and Infrequent |
| 3 | Moderate = Pain Bothersome but Can Be Ignored |
| 4 | Uncomfortable = Pain Constant but Not Too Limiting |
| 5 | Distracting = Pain Controls Attention |
| 6 | Distressing = Pain Interferes with Life |
| 7 | Intense = Pain Deteriorates Lifestyle |
| 8 | Unmanageable = Pain Very Strong Impairing Function |
| 9 | Severe = Pain Unbearable Impairing Movement |
| 10 | Debilitating = Pain Requires Immediate Attention |

Besides the pain scale presented in Table 1, there are at least ten pain scales in common use in the medical field, some of which are described below. Pain scales may generally be classified as: numerical rating scales (NRS), which use numbers to rate pain; visual analog scales (VAS) which typically ask a patient to mark a place on a scale that aligns with their level of pain; and categorical scales which use words as the primary communication tool and may also incorporate numbers, colors, or relative location to communicate pain. Most pain scales have qualitative and quantitative features.

An example of a Numerical Rating Pain Scale using ranges is represented by: 0=No

Pain; 1-3=Mild Pain; 4-6=Moderate Pain; 7-10=Severe Pain.

Another example of a pain scale is the Wong-Baker Faces (WBF) pain scale which combines pictures and numbers for pain ratings, and it is usually used for children. In WBF, six faces depict different expressions, ranging from happy to extremely upset, and each face is assigned a numerical rating between 0 (smiling) and 10 (crying). The Numerical Rating of the WBF is as follows: 0=No Pain; 2=Hurts a Little; 4=Hurts a Little More; 6=Hurts Even More; 8=Hurts Whole Lot; 10=Hurts Worst.

A FLACC pain scale assesses pain in individuals who cannot express their pain verbally. It is usually used with children who are too young or adults who are unable to communicate.

### Example 3: Pain Control Regimens

Pain control regimens may be provided for a time-point in response to a pain status report. FIG. 2D, presenting 1 tablet of Optalgin® 1000 mg is an example of a pain control regimen provided in response to pain patient's request for pain medication, shown in FIG. 2C. Optalgin is a trade name for dipyrone (sodium metamizole), a non-opioid analgesic.

Pain control regimens may also be provided as a pain control regimen based on time of the day, for example in Table 2, or based on pain status, provided in Table 3.

Table 2 presents pain medications and additional medications which may take part in a pain management regimen according to time of the day.

TABLE 2
Pain Control Regimen According to Time

| Time | Pain Medication | Additional Medication |
|---|---|---|
| 06:00 | Combination drug: | Esomeprazole |
| | Oxycodone + Acetaminophen | |
| 09:00 | Combination drug: Oxycodone | None |
| | hydrochloride + Naloxone hydrochloride | |
| 14:00 | Aceclofenac | None |
| 21:00 | Combination drug: Oxycodone | Pregabalin |
| | hydrochloride + Naloxone hydrochloride | |

Narcotic+analgesic combination drug oxycodone and acetaminophen (commonly known as the brand-name drug Percocet, for example) is used to relieve moderate to severe pain. Oxycodone is an opioid (narcotic) pain medication, and acetaminophen is a less potent pain reliever that increases the effects of oxycodone.

Combination drug oxycodone hydrochloride+naloxone hydrochloride (commercially known as Targin®, for example) is provided as a modified release drug, having oxycodone as the opioid pain medication and naloxone as a drug for counter-acting the side effects of opioids in the gut, such as constipation.

Aceclofenac (commercially known as Atofen™, for example) is a nonsteroidal anti-inflammatory drug (NSAID) analog of diclofenac, generally used for the relief of pain and inflammation, for example in rheumatoid arthritis, osteoarthritis and ankylosing spondylitis.

Esomeprazole (commercially known as Nexium®, for example) is a proton pump inhibitor that decreases the amount of acid produced in the stomach. Esomeprazole is generally used to treat symptoms of gastroesophageal reflux disease (GERD) and other conditions involving excessive stomach acid, which may be a side-effect of other conditions, or of other medications.

Pregabalin (marketed under the brand name Lyrica® among others) is a medication generally used to treat epilepsy, neuropathic pain, fibromyalgia, restless leg syndrome, and generalized anxiety disorder.

Table 3 presents alternative pain management regimens according to pain level.

TABLE 3
Pain Treatment Regimen According to Pain Level

| Pain level | | | Regimen C |
|---|---|---|---|
| (1-10 scale) | Regimen A | Regimen B | (Alternative) |
| 0 | No drugs | No drugs | Not relevant |
| 1-3 | Drug A 1x dose | Drug B 0.5x dose | Physiotherapy |
| 4-7 | Drug A 2x dose | Drug C 1x dose | Icing |
| 8-10 | Drug B | Drug C 2x dose | Meditation |

Drug A may generally be a non-opioid analgesic, such as for example acetaminophen, ibuprofen, aspirin® and the like.

Drug B may be, for example, dipyrone.

Drug C may generally be an opioid analgesic such as for example codeine, oxycodone, hydrocodone, propoxyphene, diphenoxylate, loperamide and the like.

Additional examples of drugs and dosages are provided in Table 4. The drugs listed in

Table 4 are a sample of the drugs available to the system. The list of drugs may be updated periodically with the introduction of new drugs that become available to the market and/or by exclusion of drugs that become obsolete.

TABLE 4
Drugs, Regimens, Counter-indications

| | Active | | | | | |
|---|---|---|---|---|---|---|
| | Ingredient | Drug | Max Daily | | | |
| Active Ingredient | Classification | Level | Dosage | | Remarks | |
| Acetylsalicylic Acid | | 1 | 4000 | | 1. | Recommended to take after |
| (ASA, aka Aspirin) | | | | | | meals |
| | | | | | 2. | Avoid using in combination |
| | | | | | | with Etoricoxib |
| Paracetamol | Non-Opioid | 1 | 3000-3250 | mg | | |
| | Analgesic | | | | | |
| Dipyrone | Non-Opioid | 1 | 4000 | mg | | |
| | Analgesic | | | | | |
| Buprenorphine | Weak Opioid | 2 | 40 | mg | 1. | Dosage increase can only |
| | | | | | | take place after one week |

| Tramadol | Weak Opioid | 2 | 400 | mg | 1. Should be taken in the evening |
|---|---|---|---|---|---|
| | | | | | 2. Avoid combining with SSRI¹ |
| | | | | | 3. No need to take after meals |
| Codeine | Weak Opioid | 2 | 135 | mg | |
| Oxycodone HCl | Strong Opioid | 3 | | | 1. Switch to slow release if |
| | | | | | initial dosage is not enough |
| | | | | | 2. Prescribe half the dosage to |
| | | | | | elderly and weak patients |
| | | | | | 3. Avoid usage in patients |
| | | | | | with a liver condition |
| Naloxone HCI | Opioid Antidote | 2 | | | |
| Caffeine | Not classified | 2 | | | |
| Codeine Phosphate | Weak Opioid | 2 | | | |
| Etoricoxib | NSAID/COXIB² | 2 | 120 mg/day/7 days | | 1. Avoid using in combination |
| | | | | | with ASA |
| | | | | | 2. No need to take after meals |
| Diclofenac | NSAID/COXIB | 2 | 200 | mg | |
| Naproxen | NSAID/COXIB | 2 | 1000 | mg | 1. Recommended for patients |
| | | | | | suffering from a heart |
| | | | | | condition |
| Celecoxib | NSAID/COXIB | 2 | 400 | mg | |
| Piroxicam | NSAID/COXIB | 2 | 20 | mg | |
| Lornoxicam | NSAID/COXIB | 2 | 16 | mg | |
| Ibuprofen | NSAID/COXIB | 2 | 1600-3200 | mg | 1. Recommended to take after |
| | | | | | meals |

SSRI¹—Selective serotonin reuptake inhibitors
COXIB²—COX-2 inhibitors

There is therefore provided in accordance with an embodiment of the disclosure a pain management system comprising: at least one human machine interface (HMI), operable to acquire pain data generated by a patient responsive to pain that the patient experiences and drug intake data responsive to the patient's intake of a drug for controlling the pain; at least one processor operable to process the pain and drug intake data to generate a pain management regimen; and at least one communication interface operable to support communications between an attending medical professional and the at least one HMI and/or the processor to enable the attending medical professional to access the pain and drug intake data and the pain management regimen. Optionally, the pain data comprises explicit pain data acquired based on an explicit response to pain provided by the patient. Optionally, the pain data comprises implicit pain data acquired based on an implicit response to pain exhibited by the patient. In an embodiment the pain control regimen comprises an administration regimen for administration of at least one drug to the patient.

In an embodiment the at least one processor processes the pain data to determine a degree of correlation between the explicit and implicit pain data. Optionally, the at least one processor configures the pain management regimen responsive to the degree of correlation. If the correlation is positive and the pain data indicates an increase in pain the processor may configure the pain regimen to increase administration of and/or a change in a pain control drug associated with the regimen. If the correlation is positive and the pain data indicates a decrease in pain the processor may configure the pain regimen to decrease administration of and/or a change in a pain control drug associated with the regimen.

In an embodiment if the correlation is negative, the processor may provide an indication of disagreement between the explicit and implicit pain data to at least one or any combination of more than one of the patient, an attending medical professional, a relevant other person, and/or a database configured to store the indication. Optionally the indication includes a warning of possible drug abuse.

In an embodiment the pain management system comprises a pain database having explicit and implicit pain data acquired for a plurality of people. Optionally the at least one processor generates the pain management regimen responsive to data in the pain database.

In an embodiment the at least one HMI comprises an HMI that interfaces the patient via a mobile communication device.

In an embodiment the at least one HMI comprises a patient activity HMI that acquires a measure of physical activity exhibited by the patient to provide pain data.

In an embodiment the at least one HMI comprises a patient pupil imager operable to provide a measure of pupil dilation to provide pain data.

In an embodiment the at least one HMI comprises a cardiac HMI that provides a measure of at least one or both of the patient blood pressure and/or heart rate to provide pain data.

In an embodiment the at least one HMI comprises an imaging system operable to image facial expressions of the patient to provide pain data.

In an embodiment the processor generates an alert if the explicit and implicit pain data evidences aberrant data. The alert optionally includes a warning of possible drug abuse. The alert optionally includes an indication that the patient may require intervention by the attending medical professional.

In an embodiment the at least one processor processes the data using an artificial intelligence.

Descriptions of embodiments of the invention in the present application are provided by way of example and are not intended to limit the scope of the invention. The described embodiments comprise different features, not all of which are required in all embodiments. Some embodiments utilize only some of the features or possible combinations of the features. Variations of embodiments of the invention that are described, and embodiments comprising different combinations of features noted in the described embodiments, will occur to persons of the art. The scope of the invention is limited only by the claims.

1. A pain management system comprising:
at least one human machine interface (HMI), operable to acquire explicit and implicit pain data generated by a patient responsive to pain that the patient experiences and the patient's intake of a drug for controlling the pain;
at least one processor having a neural network operable to:
process the pain and drug intake data to generate a patient managed pain management regimen; and
process the explicit and implicit pain data to detect patient abuse of the intake of drug; and
at least one communication interface operable to support communications between an attending medical professional and the at least one HMI and/or the processor to enable the attending medical professional to access the pain and drug intake data and the pain management regimen.

2-3. (canceled)

4. The pain management system according to claim 1 wherein the pain control regimen comprises an administration regimen for patient administration of at least one drug to the patient.

5. The pain management system according to claim 1 wherein the at least one processor processes the pain data to determine a degree of correlation between the explicit and implicit pain data.

6. The pain management system according to claim 5 wherein the at least one processor configures the pain management regimen responsive to the degree of correlation.

7. The pain management system according to claim 6 wherein if the correlation is positive and the pain data indicates an increase in pain the processor configures the pain regimen to increase administration of and/or a change in a pain control drug associated with the regimen.

8. The pain management system according to claim 5 wherein if the correlation is positive and the pain data indicates a decrease in pain the processor configures the pain regimen to decrease administration of and/or a change in a pain control drug associated with the regimen.

9. The pain management system according to claim 1 wherein if the correlation is negative, the processor provides an indication of disagreement between the explicit and implicit pain data to at least one or any combination of more than one of the patient, an attending medical professional, a relevant other person, and/or a database configured to store the indication.

10. The pain management system according to claim 9 wherein the indication includes a warning of possible drug abuse.

11. The pain management system according to claim 1 and comprising a pain database having explicit and implicit pain data acquired for a plurality of people.

12. The pain management system according to claim 11 wherein the at least one processor generates the pain management regimen responsive to data in the pain database.

13. The pain management system according to claim 1 wherein the at least one HMI comprises an HMI that interfaces the patient via a mobile communication device.

14. The pain management system according to claim 1 wherein the at least one HMI comprises a patient activity HMI that acquires a measure of physical activity exhibited by the patient to provide pain data.

15. The pain management system according to claim 1 wherein the at least one HMI comprises a patient pupil imager operable to provide a measure of pupil dilation to provide pain data.

16. The pain management system according to claim 1 wherein the at least one HMI comprises a cardiac HMI that provides a measure of at least one or both of the patient blood pressure and/or heart rate to provide pain data.

17. The pain management system according to claim 1 wherein the at least one HMI comprises an imaging system operable to image facial expressions of the patient to provide pain data.

18. The pain management system according to claim 1 wherein the processor generates an alert if the explicit and implicit pain data evidences aberrant data.

19. The pain management system according to claim 18 wherein the alert includes a warning of possible drug abuse.

20. The pain management system according to claim 18 wherein the alert includes an indication that the patient may require intervention by the attending medical professional

21. (canceled)

22. The pain management system according to claim 1 wherein the drug abuse comprises abuse of an opioid.

23. The pain management system according to claim 1 wherein the at least one HMI comprises a sensor that provides implicit pain data responsive to patient electrodermal activity.

24. The pain management system according to claim 1 wherein the at least one HMI comprises a sensor that provides implicit pain data responsive to monitoring patient walking and/or running motion.
